(19) United States
(12) Patent Application Publication
(10) Pub. No.: US 2006/0263448 A1
(43) Pub. Date: Nov. 23, 2006

(54) Use of extracts from roots of pelargonium sidoides and pelargonium reniforme

(75) Inventors: Egon Koch, Karlsruhe (DE); Hermann Hauer, Karlsruhe (DE); Karl-Heinz Stumpf, Karlsruhe (DE)

Correspondence Address: EDWARDS & ANGELL, LLP, P.O. BOX 55874, BOSTON, MA, 02205, US

(73) Assignee: ISO Arneimittel GmbH & CO. KG, Ettlingen (DE)

(21) Appl. No.: 10/555,189

(22) PCT Filed: Jun. 24, 2005
(86) PCT No.: PCT/EP05/06853

§ 371 Date: May 25, 2006

(30) Foreign Application Priority Data
Jul. 5, 2004 (DE) 20040324395

(51) Int. Cl. A61K 36/185

(52) U.S. Cl. 424/725

(57) ABSTRACT

The present invention relates to the use of extracts from roots of Pelargonium sidoides and/or Pelargonium reniforme for the treatment of AIDS and AIDS-associated infections caused by other viruses, bacteria, in particular mycobacteria, fungi and parasites.

The present invention relates to the use of extracts from roots of Pelargonium sidoides and/or Pelargonium reniforme for the treatment of AIDS and AIDS-associated infections caused by other viruses, bacteria, particularly mycobacteria, fungi and parasites.

About one third of the world's population suffers from tuberculosis, and this disease, which is caused by an infection with mycobacterium tuberculosis, is among the ten most important causes of death. The occurrence of infections with M. tuberculosis has dramatically increased in the past three decades due to the dissemination of the HIV/AIDS epidemic and the development of resistances against chemotherapeutic agents, particularly in Africa and South-East Asia. The weakening of the immune defense due to the infection with HIV viruses creates ideal conditions for the development of co-infections in the patients, such as with bacteria (e. g. M. tuberculosis), viruses (e. g. herpes viruses), parasites and fungi. Estimations assume that the simultaneous infection with tuberculosis is responsible for about one third of all cases of death in AIDS patients. Whereas the life expectation of AIDS patients could be improved significantly in industrialized countries due to the better hygienic and medical care and the use of antiretroviral medicaments, these medicaments are often not available to infected persons in less developed countries, last, but not least due to the high costs for these medicaments. Therefore, there is an urgent need for the development of cost-efficient alternatives for patients in these regions. Thus, the object underlying the present invention is to provide compounds and medicaments for the treatment of AIDS.

According to the present invention this object is solved by the use of extracts from Pelargonium sidoides and/or Pelargonium reniforme for the treatment of AIDS.

Pelargonium sidoides and Pelargonium reniforme are geraniacae that were traditionally used as medicaments in southern Africa. The use in public medicine comprises the treatment of diarrhea, gastrointestinal complaints, dysmenorrhea and diseases of the liver. However, the use in cases of diseases of the respiratory tract and particularly in cases of pulmonary tuberculosis is also common. The designation “Umckaloabo” derived from terms of the Zulu language for characteristic symptoms of tuberculosis, such as cough, sputum, fever and weakness of the body or incisive pains in the thorax region, is commonly used for preparations from Pelargonium sidoides/reniforme. Also in Europe the Umcka drug was widely used for the adjuvant treatment of tuberculosis under the designation “Stevens cure” at the beginning of the century. Corresponding to the traditional use, a commercially available extract from Pelargonium sidoides/reniforme is currently on the market, which is primarily used for the treatment of various acute and chronic diseases of the respiratory tract and the otolaryngologic region, such as rhinopharyngitis, tonsillitis, sinusitis and bronchitis.

It has now surprisingly been observed that extracts from the roots of Pelargonium sidoides/reniforme not only exhibit potent anti-mycobacterial and lymphoproliferative properties, but also additionally effectively inhibit the infection of human lymphocytes with the Human Immunodeficiency Virus Type 1 (HIV-1). Therefore, preparations from Pelargonium sidoides/reniforme represent interesting alternatives for the treatment of AIDS infections and AIDS-associated infections, in particular bacterial infections, such as by M. tuberculosis, atypical mycobacteria (e. g. M. avium intracellulare), Enteritis salmonellae or facultative pathogenic bacteria (e. g. streptococci, pneumococci, staphylococci or hemophilus), viral infections, such as by herpes virus (e. g. varicella-zoster, Herpes simplex), cytomegaloviruses or hepatitis viruses, infections by fungi, such as candida, cryptococcus or aspergillus, and parasitic infections, such as by Pneumocystis carinii or toxoplasms.

Extracts from geraniums or plant parts thereof can be obtained according to known preparation methods in variable compositions using solvents, such as water, methanol, ethanol, acetone and the like, and mixtures thereof at temperatures from room temperature to 60° C. under slight to rigorous mixing or by percolation within 10 min. to 25 h. Thereby, preferred extraction solvents are mixtures of ethanol and water, particularly preferred in the ratio of ethanol/water=10/90 to 15/85 (w/w). In order to further concentrate the components determining the efficacy further concentration steps can be carried out, such as liquid-liquid distribution using, for example, 1-butanol/water or ethyl acetate/water, adsorption-desorption using an ion exchanger, LH20, HP20 and other resins or chromatographic separations over RP18, silica gel and the like. If a further processing to dry extracts is desired, this is carried out in accordance with methods known per se by removing the solvent at increased temperture and/or reduced pressure or by freeze-drying.

The extracts according to the present invention can be administred in the form of powders, granules, tablets, dragees or capsules or as a solution, e. g. as directly obtained by extraction, preferably orally.

For the preparation of tablets the extract is mixed with suitable pharmaceutically acceptable adjuvants, such as lactose, cellulose, silicon dioxide, croscarmellose and magnesium stearate, and pressed into tablets that can optionally be provided with a suitable coating, for example made of hydroxymethylpropylcellulose, polyethylene glycol, colorants (e. g. titanium dioxide, iron oxide) and talcum.

The extracts according to the present invention can also be filled into capsules, optionally with addition of adjuvants, such as stabilizers, fillers and the like. The dosage is such that 2 to 1000 mg, preferably 10 to 200 mg extract are administered.

The efficacy of ethanolic aqueous extracts from Pelargonium sidoides in case of AIDS infections and AIDS-associated infections, such as tuberculosis, is supported by the following pharmacological experiments.

Assay on Anti-HIV Activity

The HIV-1 strain HTLV-III_{RF} (National Institute for Biological Standards and Control, Hertfordshire, UK) was used for the investigations. This strain is a syncytic inducing virus strain (Sl version). The amount of infective HIV particles present in the initial suspension was measured in a usual titration procedure. Thereby, the highest dilution of the virus suspension causing a cytopathologic effect (CPE) in 50% of the indicator cells (50% tissue culture infective doses per ml (TCLD50/ml)) is determined. For this determination serial dilutions of the mother suspension were prepared (1:10, 1:100, 1:1,000 etc.) and added in amounts of 50 ml to indicator cells (cf. below) in 96-well plates (in eightfold parallel tests), respectively. The read-out was carried out microscopically after 5 days by two independent examiners. C8166 cells (human lymphoid cells; National Institute for Biological Standards and Control, Hertfordshire, UK) in suspension culture were used as indicator cells.

In order to provide evidence for the “antiviral” activity eight different defined concenctrations (100; 50; 25; 12.5; 6; 3; 1.5; 0.75 μg/ml) of Pelargonium extract were mixed with a defined amount of the HIV virus (4,000 TCID₅₀/50 μl) and cells (100,000 cells/50 μl). After 5 days of incubation in an incubator at 37° C. the evaluation was effected by microscopic analysis with regard to virus-specific CPEs. The tests were carried out as a single test with eight determinations, respectively.

The determination of cytotoxicity was effected by means of an MTT test. With regard to the employed method, the procedure described above was used analogously. The evaluation of the cells was effected after 5 days. The cytotoxicity is stated as the concentration of the substance, at which about 50% of the indicator cells are destroyed by toxic influences (TC₅₀).

The calculation of the virus-inhibitory concentration (IC₅₀) was effected by means of linear regression using a computer software. The IC₅₀ value represents that concentration of a substance at which a reduction of the virus replication of 50% is achieved. The therapeutic index (TI), i. e., the ratio between TC₅₀ and IC₅₀, is additionally stated.

The extract from Pelargonium sidoides according to example 1 inhibits the virus replication half maximally at a concentration of 28 μg/ml. A value of 66 μg/ml was determined for the cytotoxic actions (IC₅₀ concentration) resulting in a therapeutic index of 2.4.

Assay on Anti-Mycobacterial Activity

To assay the anti-mycobacterial efficacy, Mycobacterium ranae that represents a non-human pathogenic mycobacterium having a similar sensitivity against chemotherapeutic agents like M. tuberculosis was used. The suspension dilution method was used to determine the minimal inhibitory concentration (MHC). The extract of Pelargonium sidoides was solved in DMSO and serially diluted to obtain the required concentrations. Pelargonium extract or the solvent was added in a volume of 10 μl to 48-well microtiter plates containing 990 μl of an M. ranae suspension (ATCC 10) in a concentration of 1-5×10⁵ CFU/ml Brain-Heart Infusion Broth (Difco, USA) per well. The maximum concentration of the solvent (DMSO) was 1%. Pelargonium extract was tested at concentrations between 100 and 0.03 μg/ml. All tests were carried out twice. The plates were incubated for 48 h at 37° C. and then the inhibition of the growth of the bacteria was evaluated visually. An MHC of 100 μg/ml was determined for the extract from Pelargonium sidoides according to example 1. Gentamicin, that was used as a positive reference compound, exhibited an MHC of 0.3 μg/ml.

Proliferation of Lymphocytes of Mice

Lymphocytes play a central role in the specific immune defense. HIV-1 viruses mainly infect CD4 lymphocytes and the loss of this lymphocyte population being important for immune reactions is of decisive importance for the pathogenesis of AIDS and mainly for the occurrence of opportunistic infections typical for this disease. Substances antagonizing the destruction of lymphocytes and their reduced regeneration are thus an efficient measure for counteracting a deficit of the cellular immune defense.

Spleen cells of male CBA/J mice (Janvier, Le Genest, France) were used for the investigations of lymphoproliferative actions of the extract from Pelargonium. After the isolation the lymphocytes were resuspended in a concentration of 2×10⁶ cells/ml in complete RPMI 1640 medium (supplemented with 10% heat-inactivated fetal bovine serum 2 mM L-glutamine, 100 U/ml penicillin, 100 μg/ml streptomycine and 50 μM 2-mercaptoethanol). Lymphocytes (10⁵ per well) were transferred to U-form microtiter plates (Greiner) and incubated in a volume of totally 200 μl complete RPMI 1640 medium in the presence of different concentrations of Pelargonium extract or solvent for 72 h in an incubator in an atmosphere of 5% of CO₂ in air. All tests were carried out in a four-fold determination. At the end of the incubation period the cells were pulsed with 18.5 kBq (37 GBq/mmol specific activity) of [methyl-³H]thymidine (Amersham, Germany) over a period of 6 h. Subsequently, the cells were collected with a cell harvester (IH 110, Berthold, Bad Wildbad) on a glass fiber filter (Type G-10, Berthold) and the incorporation of the base into the newly synthesized DNA was determined by determining the radio-activity using a proportional counter (LB 284RA, Berthold).

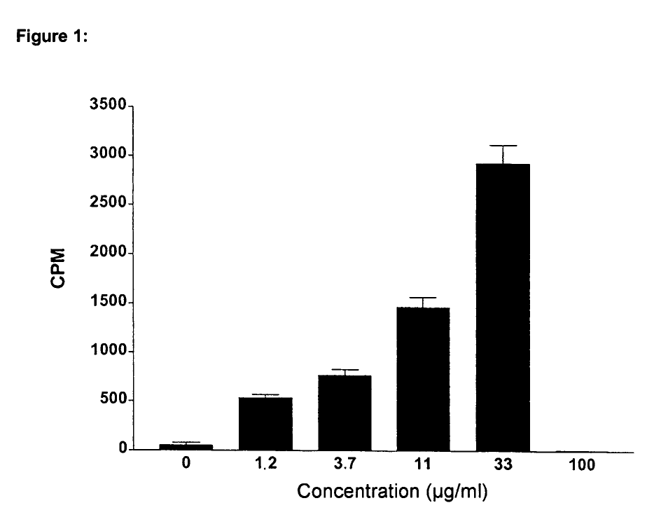

The results of the investigations with the ethanolic aqueous extract from Pelargonium sidoides according to example 2 are illustrated in FIG. 1. It can be seen therefrom that this extracts stimulates the proliferation of lymphocytes of mice in a concentration-depending manner with a maximum effect at 33 μg/ml.

FIG. 1 shows the effect of the ethanolic aqueous extract from roots of Pelargonium sidoides according to example 2 on the spontaneous proliferation of mice lymphocytes. The cell proliferation was determined by incorporating ³H-thymidines in newly synthesized DNA (counts per minute [CPM]).

### EXAMPLE 1

### Extract from Pelargonium Sidoides

Dried and ground roots of Pelargonium sidoides were extracted twice with 7 times their weight made up of ethanol/water 11/89 (w/w) at 55° C., respectively. After filtration the filtrate was concentrated and dried at 60° C. in vacuum: 106 g dry extract per kg plant material (10.6%).

### EXAMPLE 2

### Extract from Pelargonium Sidoides

About 1.15 kg ground roots from Pelargonium sidoides were extracted with about 11.5 kg ethanol/water 11/89 (w/w) at room temperature. After filtration the filtrate was concentrated at about 40° C. and freeze-dried: 86 g (7.5%) dry extract.

1-11. (canceled)

12. A method for treating a subject suffering or susceptible to AIDS, comprising administering to the subject one or more extracts from Pelargonium sidoides and/or Pelargonium reniforme.

13. The method of claim 12 wherein AIDS is associated with one or more infections by bacteria, other viruses, fungi and/or parasites.

14. The method of claim 13 wherein bacterial infection is caused by M. tuberculosis, atypical mycobacteria comprising M. avium intracellulare, Enteritis salmonellae or facultative pathogenic bacteria comprising streptococci, pneumococci, staphylococci and hemophilus.

15. The method of claim 13 wherein other viral infection is caused by herpes viruses comprising varicella-zoster and Herpes simplex, cytomegaloviruses or hepatitis viruses.

16. The method of claim 13 fungi infection is caused by fungi comprising candida, cryptococcus and aspergillus.

17. The method of claim 13 wherein parasitic infection is caused by Pneumocystis carinii or toxoplasms.

18. The method of claim 13 wherein the subject is identified as suffering from AIDS and Pelargonium sidoides and/or Pelargonium reniforme is administered to the identified subject.

19. The method of claim 13 wherein the subject is a human.

20. A pharmaceutical composition comprising one or more extracts from Pelargonium sidoides and/or Pelargonium reniforme.
